(19) United States
(12) Patent Application Publication
(10) Pub. No.: US 2021/0267710 A1
(43) Pub. Date: Sep. 2, 2021

(54) VISUAL GUIDANCE FOR ALIGNING A PHYSICAL OBJECT WITH A REFERENCE LOCATION

(71) Applicant: CARTOSENSE PRIVATE LIMITED, Maharashtra (IN)

(72) Inventors: Bhargava CHINTALAPATI, Telangana (IN); Nikhil CHANDWADKAR, Maharashtra (IN); Hem RAMPAL, Delhi (IN)

(21) Appl. No.: 17/268,748

(22) PCT Filed: Aug. 16, 2019
(86) PCT No.: PCT/IN2019/050602

§ 371 Date: Feb. 16, 2021

(30) Foreign Application Priority Data
Aug. 16, 2018 (IN) 201821030732

(51) Int. Cl. A61B 90/00; A61B 34/20; G06T 7/73; A61B 34/10

(52) U.S. Cl. CPC A61B 90/36; A61B 34/20; G06T 7/74; A61B 2034/107; A61B 2090/367; A61B 2090/363; A61B 34/10

(57) ABSTRACT

The present invention generally relates to an augmented reality, and more particularly, the present invention relates to a method for indicating alignment of a body-fixed axis (300) with a reference axis (301) of a pre-determined reference pose. In one embodiment, the method comprising: acquiring a real-time measurement of the body-fixed axis (300) predefined in a coordinate frame of the physical object (101), rendering a first surface (103) with an intersection point (304) of the reference axis (301) on a first (103) using a three-dimensional display device (100), rendering a second surface (305) at an offset from the intersection point in (304) of the reference axis (301) present on the first surface (103), rendering a plurality of set of feature graphics on the first surface (103) and the second surface (305) in one or more visual states, wherein at least one set of feature graphics of the plurality of set of feature graphics are reference feature graphics that is positionally distributed along the reference axis of the pre-determined reference axis (301), updating the positions of another set of feature graphics of the plurality of set of feature graphics based on a current position of the physical object (101), wherein the another set of feature graphics is dynamic feature graphics that is positionally distributed along the body-fixed axis (300) of the physical object (101) and modifying the visual states of the plurality of set of feature graphics based on the extent of alignment between the body-fixed axis (300) and the reference axis (301).

### FIELD OF INVENTION

The present invention generally relates to augmented reality, and particularly to provide visual assistance for performing manual tasks with a requirement of accurate alignment of an axis of a tool with a reference axis.

### BACKGROUND

Many surgical procedures, like insertion of an external ventricular drain (EVD) into a ventricle of the brain, insertion of a screw into the pedicle of a vertebra, insertion of a biopsy or ablation needle into a lung or liver tumor, etc., require visual assistance for accurately aligning the surgical instrument with a reference trajectory and steadily advancing the instrument along the trajectory without deviation. For example, a crucial step of the EVD insertion procedure involves advancing a rigid needle like instrument called a stylet through a burr hole in the skull, into a patient's brain, until it reaches anterior horn of a lateral ventricle. Typically done free-hand by a neurosurgeon using surface landmarks, inaccuracy in positioning and advancing the stylet can result in sub-optimal placement of the EVD. Corrective revision procedures are reported in 40% of the cases, each procedure adding to patient morbidity and procedural costs. Especially, in cases of distorted ventricular anatomy or unusually small ventricles, to provide means for guiding the EVD stylet safely into the ventricle is critically important. Some existing methods provide visual assistance in the form of real-time image guidance. For example, surgical navigation used for EVD insertion shows a real-time display of orthogonal Magnetic Resonance Imaging (MRI)/Computed Tomography (CT) image slices corresponding to the real-time position and the orientation of the EVD stylet. Projections of the pre-planned reference trajectory and the real-time trajectory of the EVD stylet are drawn on the image slices as graphical lines of two distinct colours.

The viewer is expected to manually adjust the EVD stylet using freehand movements to achieve overlap between the two differently coloured lines. Perfect overlap indicates accurate alignment between the real-time trajectory and the reference trajectory. However, it is cumbersome and time consuming to discover a position and orientation of the EVD stylet that achieves perfect overlap looking only at projections of the 3D space on a 2D display. This problem is additionally exacerbated because the perspective and orientation of the display does not generally match that of the surgeon, making the relation between physical hand movements and corresponding changes in the displayed lines very unintuitive. Moreover, if inadvertent movement causes even a small deviation between the two trajectories, it is cumbersome to realign them. If this inadvertent movement happens after the tissue has been penetrated, there is risk of causing damage to the tissue in the process of realigning the trajectories. Thus, there is a need for providing visual assistance for aligning an axis of a physical object with a pre-determined virtual reference trajectory such that there is a quick and an intuitive alignment between the trajectories, thereby reducing the probability of inadvertent off-trajectory movements and quick and intuitive course correction if inadvertent movements occur.

### SUMMARY

An aspect of the present invention is to address at least the above-mentioned problems and/or disadvantages and to provide at least the advantages described below.

Accordingly, in one aspect of the present invention relates to a method for indicating alignment of a body-fixed axis (300) with a reference axis (301) of a pre-determined reference pose, the method comprising: acquiring a real-time measurement of the body-fixed axis (300) predefined in a coordinate frame of the physical object (101), rendering a first surface (103) with an intersection point (304) of the reference axis (301) on a first surface (103) using a three-dimensional display device (100), rendering a second surface (305) at an offset from the intersection point (304) of the reference axis (301) present on the first surface (103), rendering a plurality of set of feature graphics on the first surface (103) and the second surface (305) in one or more visual states, wherein atleast one set of feature graphics of the plurality of set of feature graphics are reference feature graphics that is positionally distributed along the reference axis of the pre-determined reference pose (301), updating the positions of another set of feature graphics of the plurality of set of feature graphics based on a current position of the physical object (101), wherein the another set of feature graphics is dynamic feature graphics that is positionally distributed along the body-fixed axis (300) of the physical object (101) and modifying the visual states of the plurality of set of feature graphics based on the extent of alignment between the body-fixed axis (300) and the reference axis (301).

Another aspect of the present invention relates to a visual guidance system for indicating alignment of a physical object (101) with a reference axis (301) of a pre-determined reference pose, the visual guidance system comprising one or more processors coupled and configured with components of the visual guidance system for indicating alignment of the physical object (101) with the pre-determined reference axis (301), the system comprising: a three-dimensional display device (100) for rendering a first surface (103) with an intersection point (304) of the reference axis (301) on a first surface(103), a physical object (101) for performing an action, a tracking system (102) for tracking the position and orientation of the physical object (101), memory device comprising the reference axis (301) of the pre-determined reference pose, the three dimensional display device (100) for rendering a body-fixed axis (300) based on the tracked position and orientation of the physical object and a plurality of set of feature graphics on the first surface (103) and the second surface (305) in one or more visual states, wherein atleast one set of feature graphics of the plurality of set of feature graphics is positionally distributed along the pre-determined reference axis (301) and the three dimensional display device (100) for rendering modified visual states of the plurality of set of feature graphics based on the extent of alignment between the body-fixed axis (300) and the reference axis (301).

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF DRAWINGS

The above and other aspects, features, and advantages of certain exemplary embodiments of the present invention will be more apparent from the following description taken in conjunction with the accompanying drawings in which:

FIG. 1. illustrates components of a visual guidance system used by a user, such as, a surgeon during the intervention.

FIG. 2. illustrates an optically tracked physical object, the user would advance in a body part such as a patient's brain.

FIG. 3. illustrates the view of a user through a tracked three dimensional display device, where an augmented reality visualization is rendered by an augmented reality display device worn by the user.

FIG. 4A. illustrates the state of the augmented reality visualization when axis of the physical object is not aligned with a pre-determined virtual reference trajectory.

FIG. 4B. illustrates the state of the augmented reality visualization when axis of the physical object is partially aligned with a pre-determined virtual reference trajectory.

FIG. 4C. illustrates the state of the augmented reality visualization when axis of the physical object is accurately aligned with a pre-determined virtual reference trajectory.

FIGS. 5A-5B, illustrates the dynamic features required in the visualization to align a spatially tracked physical object.

FIGS. 6A-6E illustrates the modification of visual states of the reference feature graphics and the dynamic feature graphics for aligning a physical object.

FIG. 7 illustrates the dynamic features required in the visualization to align a physical object.

FIG. 8 illustrates the modification of visual states of the reference and the dynamic feature graphics for aligning a physical object.

FIG. 9 illustrates a method for indicating alignment of a physical object with a pre-determined virtual reference trajectory.

Persons skilled in the art will appreciate that elements in the figures are illustrated for simplicity and clarity and may have not been drawn to scale. For example, the dimensions of some of the elements in the figure may be exaggerated relative to other elements to help to improve understanding of various exemplary embodiments of the present disclosure. Throughout the drawings, it should be noted that like reference numbers are used to depict the same or similar elements, features, and structures.

### DETAILED DESCRIPTION OF THE DRAWINGS

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions are omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention are provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

By the term “substantially” it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic is intended to provide.

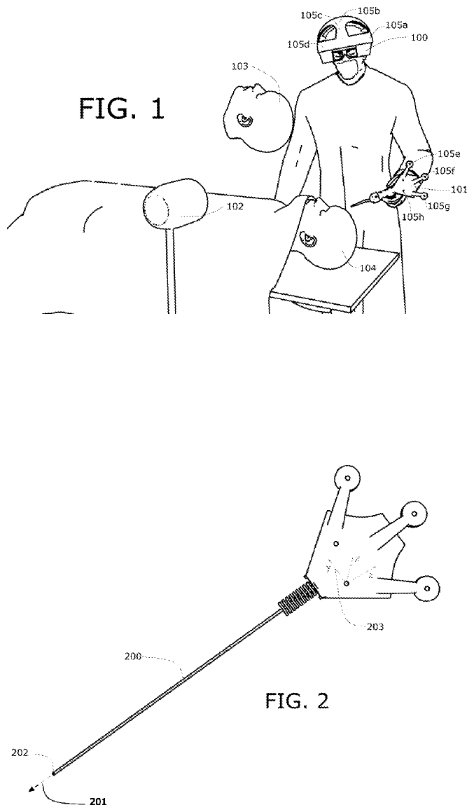

FIGS. 1 through 9, discussed below, and the various embodiments used to describe the principles of the present disclosure in this patent document are by way of illustration only and should not be construed in any way that would limit the scope of the disclosure. Those skilled in the art will understand that the principles of the present disclosure may be implemented in any suitably arranged communications system. The terms used to describe various embodiments are exemplary. It should be understood that these are provided to merely aid the understanding of the description, and that their use and definitions, in no way limit the scope of the invention. Terms first, second, and the like are used to differentiate between objects having the same terminology and are in no way intended to represent a chronological order, unless where explicitly stated otherwise. A set is defined as a non-empty set including at least one element.

A virtual three dimensional 3D environment is an immersive computer-generated environment which the user can perceive and interact with. Augmented reality AR is a technology that is used to generate and present a virtual 3D environment, where the user perceives computer generated graphics to be a part of the real environment. One of the applications of AR is in providing visual guidance, in the form of graphical elements overlaid on the tools used for performing complex or safety critical tasks. These graphical elements perceived by the user as physical extensions of the tools enhance hand-eye co-ordination as all directions perceived by the user in a physical space map to same set of directions in the virtual 3D environment. The visual guidance is provided to the user through a three dimensional display device, which could be a stereoscopic optical or video see-through head mounted display, a head-mounted virtual reality display, or any other three dimensional display device such as a light-field or holographic display—not necessarily head-mounted.

AR visual guidance can assist several medical applications where an instrument must access a lesion in the patient without impairing healthy anatomy. The intended position and orientation of the instrument is its reference pose the user wants to achieve. Reference pose could be a linear trajectory that can be used for advancing EVD stylets, for setting up biopsy needle holders etc. Reference pose could be a linear trajectory with preferred depth along the trajectory used for introducing biopsy needles, inserting K-wires into vertebrae, fine needle aspiration, introducing ablation needles, dispensing bone cement for vertebroplasty, positioning electrodes for deep-brain stimulation, administering nerve blocks, positioning orthopedic implants etc. Reference pose could be a linear trajectory with preferred depth along the trajectory and orientation about the trajectory used for positioning imaging equipment, positioning instrument holders etc. In these cases, the linear trajectory used to define the reference pose is the reference axis, the preferred depth along the trajectory to be achieved by the instrument is captured by the reference point and the preferred orientation about the trajectory is captured by the reference direction.

AR visual guidance can assist non-medical applications where an object must be precisely positioned and oriented relative to another. Reference pose containing only linear trajectory could be used for positioning visual inspection instrument relative to specimens being inspected. Reference pose containing a linear trajectory with preferred depth along the trajectory could be used on the assembly line to guide a mechanical arm driving fasteners into a chassis. Reference pose containing a linear trajectory with preferred depth and orientation about the trajectory can be used to guide a glue dispensing mechanism to follow a complex lip-groove contour on a product. In these cases, the instrument direction used to define the reference pose is the reference axis, the preferred depth along the trajectory to be achieved by the instrument is captured by the reference point and the preferred orientation about the trajectory is captured by the reference direction.

FIG. 1 exemplarily illustrates a visual guidance system configuration that provides augmented-reality based visual guidance. The visual guidance system, for example, is used in context of inserting an external ventricular drain EVD, a common procedure in neurosurgery. The visual guidance system comprises components such as the three dimensional display device 100, a physical object 101, and a tracking system 102. A user, that is a surgeon, wears the three dimensional display device 100 holding a physical object 101, that which are optically tracked by a tracking system, that is, a camera 102, by imaging the active LED markers 105 rigidly attached to them. The physical object 101 and the three dimensional display device 100 can also be tracked using other technology such as electromagnetic tracking, fiber optic shape sensing, laser tracking, or an articulating arm. A virtual patient model, that is a first surface, 103 is part of the virtual 3D environment that is presented to the surgeon through the three dimensional display device 100. The reference pose of the physical object 101 is pre-operatively determined in the coordinate frame of the virtual patient model 103. A registration step is performed between the virtual patient model 103 and a real patient, that is a real environment object, 104, to estimate the transform between the tracking system coordinate frame and the coordinate frame of virtual patient model 103. After the registration, a virtual instrument that replicates the movements of the physical object 101 relative to the virtual patient model 103, can be added to the virtual 3D environment rendered by the three dimensional display device 100.

The virtual 3D environment provides one or more user interface features that allow the surgeon to use the physical object 101 for rotating and scaling the virtual patient model 103. In an embodiment, the orientation of the virtual patient model 103 is the same as that of the real patient 104. Displaying the virtual patient model 103 in the same orientation enhances hand-eye co-ordination as all directions perceived by the viewer, that is the surgeon in the physical space map to the same set of directions in the virtual 3D environment. To present the virtual 3D environment to the user in the user's perspective, the user's eye position relative to head mounted display 100 is assumed to be a constant. In an embodiment, the position as well as the orientation of the virtual model 103 is the same as that of the real patient 104, this requires estimating the user's eye position relative to the three dimensional display unit 100 using a calibration step such as single point active alignment method SPAAM. In another embodiment, the user's eye position relative to the three dimensional display device is tracked in real-time and used as the projection point.

Upon locking of both the position and orientation, the virtual patient model 103 is perceived to be completely overlapped with the real patient 104. This is the most intuitive mode of visualization for the highest-accuracy hand-eye coordination, as it enables true augmentation where virtual objects behave as graphical extensions of the real objects.

FIG. 2A exemplarily illustrates an optically tracked physical object 106 that is a stylet. 200, with the stylet axis 201 and stylet tip 202 defined and pre-calibrated in the coordinate frame 203 of the stylet 200. During the intervention, the surgeon advances the stylet 200 along the stylet axis 201 into the tissue. A body-fixed axis is chosen in the coordinate frame 203 depending on intended use. For EVD insertion, the body-fixed axis is considered as the stylet axis 201. The real-time position and orientation of the body-fixed axis can be directly received as a measurement from the tracking system 102. The real-time position and orientation of the body-fixed axis can be estimated by applying a pre-defined transformation to the position and orientation measurement received from the tracking system 102 of coordinate frame 203 of the stylet 200. The body-fixed axis is visualized in the virtual 3D environment as a part of the virtual instrument that mimics the movements of the optically tracked stylet 200.

The visual guidance system comprises one or more processors and one or more computer readable storage medium. The one or more processors are coupled and configured with the components of the visual guidance system, that is the three dimensional display device 100, the tracking system 102, and the physical object 101 for indicating alignment of an axis of the physical object 101 with the pre-determined reference axis 301. The methods and algorithms corresponding to the visual guidance system may be implemented in a computer readable storage medium appropriately programmed for general purpose computers and computing devices. Typically the processor, for e.g., one or more microprocessors receive instructions from a memory or like device, and execute those instructions, thereby performing one or more processes defined by those instructions. Further, programs that implement such methods and algorithms may be stored and transmitted using a variety of media, for e.g., computer readable storage media in a number of manners. A “processor” means any one or more microprocessors, Central Processing Unit CPU devices, computing devices, microcontrollers, digital signal processors or like devices.

The term “computer-readable storage medium” refers to any medium that participates in providing data, for example instructions that may be read by a computer, a processor or a like device. Such a medium may take many forms, including but not limited to, non-volatile media, volatile media. Non-volatile media include, for example, optical or magnetic disks and other persistent memory volatile media include Dynamic Random Access Memory DRAM, which typically constitutes the main memory. A transmission media include coaxial cables, copper wire and fiber optics, including the wires that comprise a system bus coupled to the processor and the computer readable storage media for providing the data. Common forms of computer-readable storage media include, for example, a floppy disk, a flexible disk, hard disk, magnetic tape, any other magnetic medium, a Compact Disc-Read Only Memory CD-ROM, Digital Versatile Disc DVD, any other optical medium, punch cards, paper tape, any other physical medium with patterns of holes, a Random Access Memory RAM, a Programmable Read Only Memory PROM, an Erasable Programmable Read Only Memory EPROM, an Electrically Erasable Programmable Read Only Memory EEPROM, a flash memory, any other memory chip or cartridge, a carrier wave as described hereinafter, or any other medium from which a computer can read. In general, the computer-readable programs may be implemented in any programming language. Some examples of languages that can be used include C, C++, C#, or JAVA. The program will use various security, encryption and compression techniques to enhance the overall user experience. The software programs may be stored on or in one or more mediums as an object code. A computer program product comprising computer executable instructions embodied in a computer-readable medium comprises computer parsable codes for the implementation of the processes of various embodiments.

The method and the visual guidance system disclosed herein can be configured to work in a network environment comprising one or more computers that are in communication with one or more devices via a network. In an embodiment, the computers communicate with the devices directly or indirectly, via a wired medium or a wireless medium such as the Internet, a local area network LAN, a wide area network WAN or the Ethernet, a token ring, or via any appropriate communications mediums or combination of communications mediums. Each of the devices comprises processors, examples of which are disclosed above, that are adapted to communicate with the computers. In an embodiment, each of the computers is equipped with a network communication device, for example, a network interface card, a modem, or other network connection device suitable for connecting to a network. Each of the computers and the devices executes an operating system, examples of which are disclosed above. While the operating system may differ depending on the type of computer, the operating system provides the appropriate communications protocols to establish communication links with the network. Any number and type of machines may be in communication with the computers.

In an embodiment, the visual guidance system for indicating alignment of the physical object 101 with the reference axis 301 of the pre-determined reference pose, the visual guidance system comprising one or more processors coupled and configured with components of the visual guidance system for indicating alignment of the physical object 101 with the pre-determined reference axis 301. The system comprises the three-dimensional display device 100 for rendering the first surface 103 with the intersection point 304 of the reference axis 301 on the first surface 103, the physical object 101 for performing an action, the tracking system 102 for tracking the position and orientation of the physical object 101, the memory device comprising the reference axis 301 of the pre-determined reference pose. The three dimensional display device 100 renders the body-fixed axis 300 based on the tracked position and orientation of the physical object and the plurality of set of feature graphics on the first surface 103 and the second surface 308 in one or more visual states, wherein atleast one set of feature graphics of the plurality of set of feature graphics is positionally distributed along the pre-determined reference pose 301. The three dimensional display device 100 for renders modified visual states of the plurality of set of feature graphics based on the extent of alignment between the body-fixed axis 300 and the reference axis 301. In an embodiment, rendering is one of providing and/or displaying the first surface and the second surface. The set of feature graphics of the plurality of feature graphics along the reference axis 301 are the first reference feature graphic 302 and the second reference feature graphic 307 and the another set of feature graphics of the plurality of set of feature graphics are the first dynamic feature graphic 310 and the second dynamic feature graphic 312. The position and orientation of the first surface 103 is same as position and orientation of the real environment object 104. The second surface 305 rendered by the three dimensional display device 100 is transparent. The tracking system 102 also tracks the position and orientation of the three dimensional display device 100 in real time.

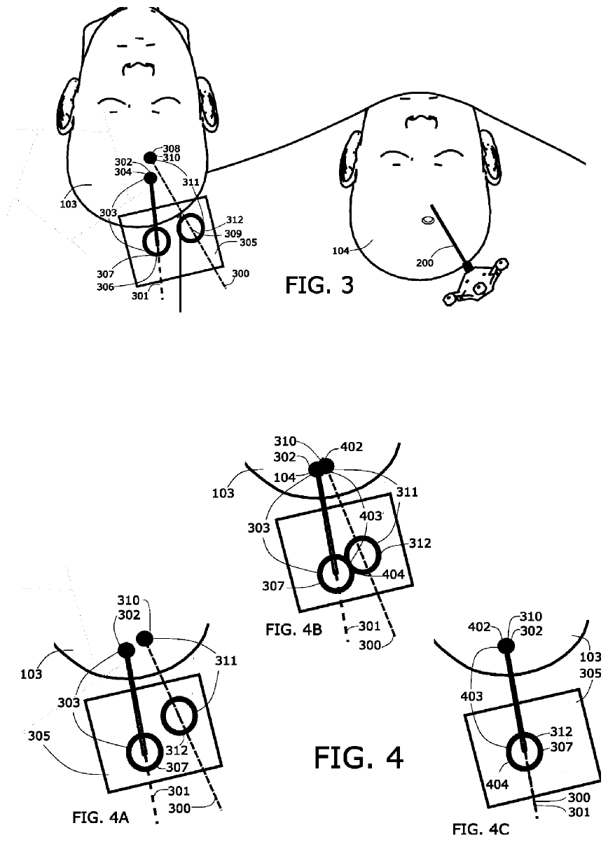

FIG. 3 exemplarily illustrates a view through the three dimensional display device 100 with augmented reality AR graphical elements for aligning an object 200 like an EVD stylet, against a reference pose 301 which is a linear trajectory. The body-fixed axis 300 is the axis 201 of the stylet. The reference axis 301 is along the linear trajectory that the user prefers to advance the instrument into a body part of the patient, that is the patient's brain. The reference axis 301 along the linear trajectory ensures that when the body-fixed axis 300 aligns with the reference axis 301, the stylet 200 advances along the desired linear trajectory. The first surface 103 intersecting the reference axis 301 at the intersection point 304, is the head surface of the virtual patient model 103. A first reference feature graphic 302 could be any symmetric shape drawn on the first surface 103. In an embodiment, first reference feature graphic 302 is a filled circle, centered at the intersection point 304. The initial visual state 303 of the first reference feature graphic 302 is red color 303. The second surface 305 is a virtual plane 305, placed at an offset from the virtual patient model 103, along the reference axis 301. The second surface 305 intersects the reference axis 301 at a intersection point 306. The second reference feature graphic could be any symmetric shape drawn on the second surface 305, for example, an annular ring 307, centered at the intersection point 306. The initial visual state of the second reference feature graphic is the red color 303. The initial visual state of the first and the second reference feature graphic is shared by the means of a common color 303. Although the first and the second reference feature graphics have the same colors here, they could have different colors.

The line 300 is the body-fixed axis of the physical object 200, intersecting the first surface 103 at the intersection point 308 and intersecting the second surface 305 at the intersection point 309. In real-time as the user moves the physical object 200, the body-fixed axis 300, the intersection point 308 and the intersection point 309 is updated.

The first dynamic feature graphic 310 could be any symmetric shape drawn on the first surface 103 coupled to the intersection point 308. In an embodiment, first dynamic feature graphic 310 is a filled circle 310 centered about the intersection point 308. The initial visual state of the first dynamic feature graphic is the yellow color 311. The second dynamic feature could be any symmetric shape drawn on the second surface 305 coupled to the intersection point 309, for example, an annular ring 312 centered at the intersection point 309. The initial visual state of the second dynamic feature 312 is the yellow color, second visual state, 311. The initial visual state of the first and the second dynamic feature graphic is shared by the color 311. Although the first and the second dynamic feature graphics have the same colors here, they could also have different colors. The first dynamic feature graphics 310 and the second dynamic feature graphics 312 have the same dimensions as the first reference feature graphics 302 and the second reference feature graphics 307 respectively.

There are two features of the visualization that enhance the viewer's hand-eye coordination. Firstly, rendering the virtual patient model 103, the body-fixed axis 300 to the user in a perspective and orientation close to the close to the perception of the physical object 200 and the real patient 104. Secondly, ensuring the relative pose between the physical object 200 and the real patient 104 is the same as the relative pose between the body-fixed axis 300 and the virtual patient model 103, thereby enabling the user to perceive the body-fixed axis 300 as mimicking the motions of the physical object 200 in the real environment.

FIGS. 4A-4C exemplarily illustrate different trajectory alignment guidance features. The FIGS. 4A-4C show the distinct appearances of the different cases of no alignment, partial alignment and perfect alignment respectively. The figures show the modification of visual states of the reference and the dynamic feature graphics as the user aligns the body-fixed axis 300 with the reference axis 301. As the alignment error between the body-fixed axis 300 and the reference axis 301 decreases, the area of overlap between the reference and the dynamic feature graphics increases. In response to the decreased error between the body-fixed axis 300 and the reference axis 301, visual states of the areas of overlap are modified.

FIG. 4A illustrates the reference axis 301 and the body-fixed axis 300 being unaligned. The reference feature graphics 302 and 307 are in the initial visual state, first visual state 303. The dynamic feature graphics 310 and 312 are in the initial visual state, second visual state 311. FIG. 4B illustrates the visual state of the graphics when the body-fixed axis 300 and the reference axis 301 are partially aligned due to which the reference feature graphics have areas of overlap 402 and 404 with the corresponding dynamic feature graphics on the first surface 103 and the second surface 305 respectively. The areas of overlap 402 and 404 are in modified visual state which is the third visual state, that is green color 403.

FIG. 4C illustrate the visual state of the graphics with complete alignment. The reference feature graphics and the dynamic feature graphics are exactly overlaid on top of each other on both the first surface 103 and the second surface 305. In the event of complete alignment, the first surface 103 has only one first feature graphic 402 and the second surface 305 has only one second feature graphic 404, both in a modified visual state, that is the third visual state, that is in green color 403. Any deviation from the above mentioned third visual state 403 indicates an onset of misalignment between the body-fixed axis 300 and the reference axis 301. In an embodiment, the visual states of the feature graphics are shapes. For example, the first visual state of the first reference feature graphics and the first dynamic feature graphics are rendered in a square shape, the first visual state of the second reference and the second dynamic feature graphics are rendered in a circular shape. The modification of the visual state upon complete alignment is another shape, for example, a triangle. In an embodiment, an angular difference between the pre-determined reference axis 301 and the body-fixed axis 300 is displayed. In an embodiment, the reference feature graphics and the dynamic feature graphics are dots, annuli, spheres, annular arcs, or a combination thereof.

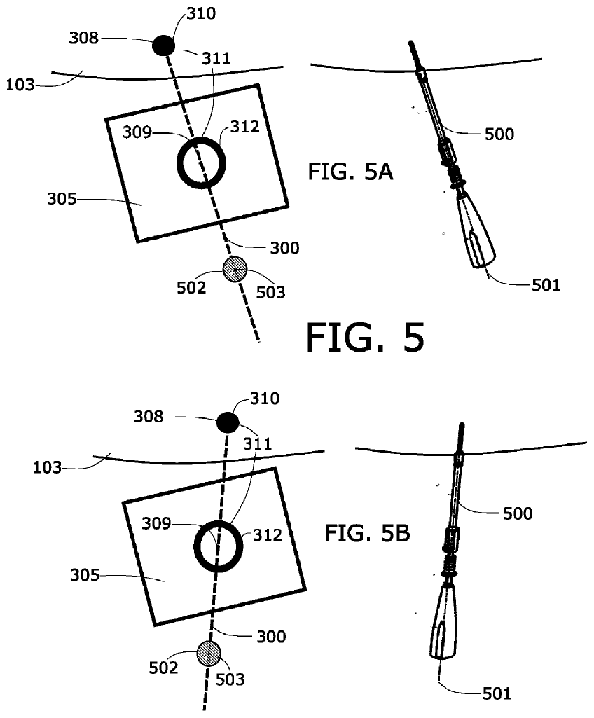

FIGS. 5A-5B, illustrates the dynamic features required in the visualization to align a spatially tracked physical object 101 for example, a cannulated needle 500 for K-wire insertion, against a linear instrument trajectory and advance the cannulated needle 500 along the instrument trajectory to a fixed depth. The body-fixed axis 300 of the cannulated needle 500 is the axis 501. The body-fixed point 503 could be any pre-determined point along the body-fixed axis 300. The body-fixed axis 300 of the spatially tracked object, intersects the first surface 103 at the intersection point 308 and intersects the second surface 305 at the intersection point 309. The dynamic feature graphics, that is the first dynamic feature graphic 310 and the second dynamic feature graphic 312 on the first surface 103 and second surface 305 are coupled to the intersection point 308 and the intersection point 309 respectively. Here the dynamic feature graphics 310 and 312 are centered about the intersection points 308 and 309 respectively. The dynamic feature graphics are in the initial visual state, that is a second visual state, for example, in yellow color 311. A third dynamic feature can be any shape coupled to the body-fixed point 503. The third dynamic feature here is a sphere 502, that functions as a depth indicator. The sphere 502 is centered about the point 503, that is at a fixed distance from the tip of the spatially tracked physical object, that is the cannulated needle 500 along the body-defined axis 300. It is not necessary for the third dynamic feature graphic 502 to be the same visual state as the first and the second dynamic feature graphics.

FIGS. 5A-5B illustrate that the body-fixed axis 300, the intersection point 308, the intersection point 309, the point 503 , the first dynamic feature 310, the second dynamic feature 312 and the third dynamic feature 502 are updated in real-time as the user moves the physical object 500.

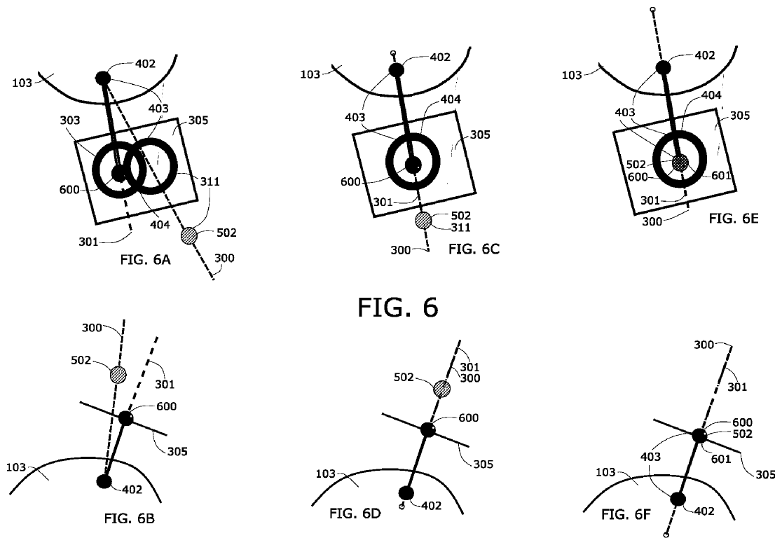

FIGS. 6A-6E illustrates the modification of visual states of the reference feature graphics and the dynamic feature graphics for aligning a physical object, for example, the cannulated needle 500 for K-wire insertion, against a reference pose that is a linear trajectory with a preferred depth along the trajectory. The reference axis 301 is along linear trajectory along which the user wants to place the K-wire in for example, a patient's vertebra, and a reference depth indicator. The third reference feature graphic 600 is centered about the reference point 309 chosen to control the bore depth of the K-wire. In this case, the second intersection point 309 is also the reference point, chosen such that when the body-fixed axis 300 aligns with the reference axis 301 and the third dynamic feature graphic 502 aligns with the third reference feature graphic 600, the user has inserted the K-wire along the desired trajectory and at the desired depth.

The extent of alignment is governed by the alignment between the body-fixed axis 300 with the reference axis 301 and by the alignment of the third reference feature graphic 600 with the third dynamic feature graphic 502. As the alignment error between the body-fixed axis 300 and the reference axis 301 decreases, the area of overlap between the reference feature graphics and the dynamic feature graphics on the first surface 103 and second surface 105 increases. The decreased error between the body-fixed axis 300 and the reference axis 301 leads to modification of visual states of the areas of overlap. As the spatially tracked physical object 101 advances along the reference axis 301 and approaches the intended depth, the distance between the reference point and the body-fixed point decreases, thereby the distance between the third reference feature graphics 600 and the third dynamic feature graphics 502 decreases. In response to achieving the intended depth along reference axis within a threshold, both the third reference feature graphic 600 and third dynamic feature graphic 502 are brought into the same modified visual state

FIG. 6A illustrates visual state of the feature graphics when the reference axis 301 and the body-fixed axis 300 are partially aligned, due to which the reference feature graphics have areas of overlap 402, 404 with the dynamic feature graphics on the first surface 103 and the second surface 305. The areas of overlap 402, 404 are in a modified visual state, that is a third visual state, that is green color 403. The third reference feature graphic 600 and the third dynamic feature graphic 502 are not aligned and retain their initial first visual state, that is, red color 303 and the second visual state, that is the yellow color 311 respectively. FIG. 6B illustrates the side view of the visualization as illustrated and described in the FIG. 6A, with partial alignment between the reference axis 301 and the body-fixed axis 300 and the third reference feature graphic 600 not being aligned with the third dynamic feature graphic 502.

FIG. 6C illustrates the visual state of the feature graphics when there is partial alignment. The reference axis 301 and the body-fixed axis 300 are completely aligned, however the third reference feature graphic 600 and the third dynamic feature graphic 502 are not aligned. The reference feature graphics and the dynamic feature graphics are overlaid on top of each other on both the surfaces, the first surface 103 has one first feature graphic 402 and the second surface 305 has one second feature graphic 404, both in a modified visual state which is the green color 403. The third reference feature graphic 600 and the third dynamic feature graphic 502 are not aligned. FIG. 6D illustrates the side view of the visualization illustrated and described in the FIG. 6C, with the reference axis 301 and the body-fixed axis 300 aligned while the third reference feature graphic 600 and the third dynamic feature graphic 502 are not aligned.

FIG. 6E illustrates the visual state of the graphics when there is complete alignment. The reference feature graphics and the dynamic feature graphics are exactly overlaid on top of each other on both the surfaces 103 and 305. The third dynamic feature graphic 502 is close to the third reference feature graphic 600 within a threshold. In an event of complete alignment, the first surface 103 has a single first feature graphic 402, the second surface 305 has a single second feature graphic 404 and a single third feature graphic 601 in a modified visual state, that is the third visual state, that is the green color 403. FIG. 6F illustrates the side view of the visualization illustrated and described in the FIG. 6E with the reference axis 301, the third reference feature graphic 600 aligned with the body-fixed axis 300, the third dynamic feature graphic 502 respectively. Any deviation from the third visual state indicates an onset of misalignment between the spatially tracked physical object 500 and the reference pose.

In an embodiment, the first surface 103 is one of transparent, translucent, and opaque or a combination thereof. In an embodiment, the real environment object 104 is a patient or any body part of the patient. In another embodiment, the real environment object 104 is any physical object that exists in a real world. In an embodiment, the first surface 103 is a three dimensional visualization of the real environment object 104. In another embodiment, the first surface 103 is a plane rendered at an offset from a real environment object 104. In an embodiment, an action is a medical procedure. In another embodiment, an action is a non medical procedure. In an embodiment, the first visual state 303 and the second visual state 315 are distinct and the third visual state 403 is distinct from the first visual state 303 and the second visual state 315. In another embodiment, the first visual state 303 and the second visual state 315 are not distinct and the third visual state 403 is distinct from the first visual state 303 and the second visual state 315.

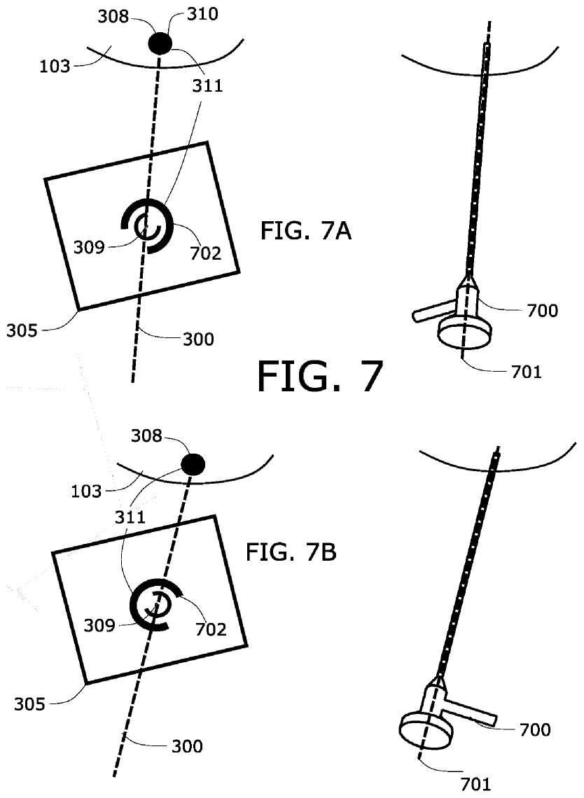

FIG. 7 illustrates the dynamic features required in the visualization to align a physical object like a neuro-endoscope 700, against a linear trajectory and advance it retaining an orientation about the trajectory. The body-fixed axis 300 of the device is the axis 701 of the neuro-endoscope 700. The body-fixed direction could be any pre-defined direction non-parallel to the body-fixed axis 300. The body-fixed axis 300 of the physical object 700, intersects the first surface 103 at the intersection point 308 and intersects the second surface 305 at the intersection point 309. A first dynamic feature graphic 310 is drawn on the first surface 103 in the initial visual state of the yellow color 311. A dynamic body-fixed direction indicator, that could be any shape, that is, asymmetric about the body-fixed axis, such as an off-centered circle, annular arc, etc. An asymmetric second dynamic feature graphic 702 is drawn on the second surface 305 coupled to the intersection point 309. Here it is centered about the intersection point 309 in the initial visual state of the yellow color 311. The azimuth of the body-fixed direction on the second surface 305 is used to orient the asymmetric second dynamic feature 702. It is not necessary for asymmetric second dynamic feature graphic 502 to be of the same visual state as the first dynamic feature graphic. FIGS. 7A-7B illustrate that the body-fixed axis 300, the intersection point 308, the intersection point 309, the first dynamic feature graphic 302, the asymmetric second dynamic feature graphic 702 are updated in real-time as the user moves the physical object 700.

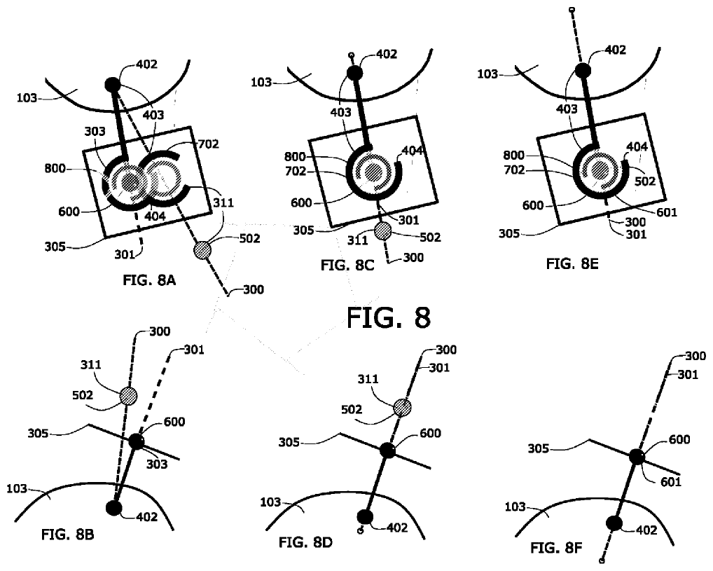

FIG. 8 illustrates the modification of visual states of the reference and the dynamic feature graphics for aligning a physical object like an ultrasound probe, against a reference pose which is a linear trajectory with a preferred depth along the trajectory and a preferred orientation about the trajectory. The body-fixed axis 300 of the ultrasound probe is chosen to lie in the imaging plane of the transducer. The body-fixed direction is a pre-defined direction that it is non parallel to the body-fixed axis. The body-fixed point 503 is a pre-determined point along the body-fixed axis. The reference axis 301 is along a linear trajectory the user wants to hold the ultrasound probe along. The reference point 309 and the asymmetric second reference feature graphic 800 are chosen such that when the body-fixed axis 300, the third dynamic feature graphic 502 and the asymmetric second dynamic feature graphic 702 align with the reference axis 301, the third reference feature graphic 600 and the asymmetric second reference feature graphic 800 respectively, the user has positioned and oriented the ultrasound transducer to precisely image the intended plane of an organ. The extent of alignment is governed by the alignment of the body-fixed axis 300, the asymmetric second dynamic feature graphic 702, the third dynamic feature graphic 502 with the reference axis 301, the asymmetric second reference feature graphic 800, the third reference feature graphic 600 respectively. The area of overlap between the reference and the dynamic feature graphics on both the surfaces increases as the alignment error between the body-fixed axis 300 and the reference axis 301 decreases and the angular error between the asymmetric second dynamic feature graphic 702 and the asymmetric second reference feature graphic 800 decreases. In response to the decreased alignment error, visual states of the areas of overlap on both the surfaces 103 and 305 are modified. As the physical object advances along the reference axis and approaches the intended depth, the distance between the reference point and the body fixed point decreases, thereby the distance between the third reference feature graphic 600 and the third dynamic feature graphic 502 decreases. In response to achieving the intended depth along reference axis within a threshold, both the third reference feature graphic 600 and third dynamic feature graphic 502 are brought into the same modified visual state.

FIG. 8A illustrates the visual state of the graphics when the reference axis 301 and the body-fixed axis 300 are partially aligned, because of which the reference feature graphics have areas of overlap 402, 404 with the dynamic feature graphics on both the surfaces 103, 305. The areas of overlap 402, 404 are in a modified visual state which is the green color 403. The reference third feature graphic 600 and the dynamic third feature graphic 502 are not aligned. FIG. 8B illustrates the side view of the visualization described in FIG. 8A, with partial alignment between the reference axis 301 and the body-fixed axis 300 and the third reference feature graphic 600 not being aligned with the third dynamic feature graphic 502.

FIG. 8C illustrates the visual state of the graphics when there is partial alignment. The reference axis 301 and the body-fixed axis 300 are completely aligned. The asymmetric second reference feature graphic 800 and the asymmetric second dynamic feature graphic 702 are completely aligned. The reference feature graphics and the dynamic feature graphics are exactly overlaid on top of each other on both the surfaces 103 and 305, the first surface 103 has one first feature graphic 402 and second surface 305 has one second feature graphic 404, both in a modified visual state which is the green color 403. The third reference feature graphic 600 and the third dynamic feature graphic 502 are not aligned. FIG. 8D illustrates the side view of the visualization described in FIG. 8C, with the reference axis 301 and the body-fixed axis 300 aligned and the third reference feature graphic 600 and the third dynamic feature graphic 502 not aligned.

FIG. 8E illustrates the visual state of the graphics when there is complete alignment. The reference feature graphics and the dynamic feature graphics are exactly overlaid on top of each other on both the surfaces 103 and 305. The third dynamic feature graphic 502 is close to the third reference feature graphic 600 within a threshold. In the event of complete alignment, the first surface 103 has only one first feature graphic 402, the second surface 305 has only one asymmetric second feature graphic 404 and only one third feature graphic 601 all of them in a modified visual state which is the green color 403. Referring to FIG. 8F, which shows the side view of the visualization described in FIG. 8E, with the reference axis 301, the reference asymmetric feature graphic 800, the third reference feature graphic 600 are aligned with the body-fixed axis 300, the dynamic asymmetric feature graphic 702, the dynamic third feature graphic 502 respectively. Any deviation from this visual state indicates an onset of misalignment between the physical object and its reference pose.

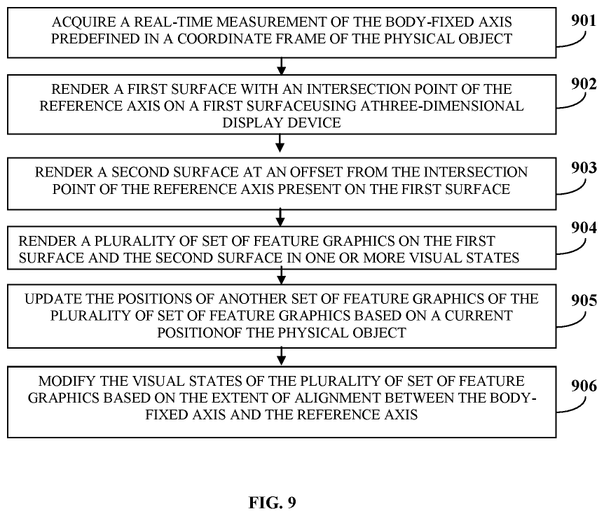

FIG. 9 illustrates a method for indicating alignment of a body-fixed axis 300 with a reference axis 301 of a pre-determined reference pose. The method comprising acquiring 901 a real-time measurement of the body-fixed axis 300 predefined in the coordinate frame of the physical object 101 and rendering 902 the first surface 103 with the intersection point 304 of the reference axis 301 on the first surface 103 using the three-dimensional display device 100. The method further comprising rendering 903 the second surface 305 at an offset from the intersection point 304 of the reference axis 301 present on the first surface 103. In an embodiment, rendering is one of providing and/or displaying the first surface and the second surface

The method further comprises rendering 904 a plurality of set of feature graphics on the first surface 103 and the second surface 305 in one or more visual states, wherein atleast one set of feature graphics of the plurality of set of feature graphics are reference feature graphics that is positionally distributed along the reference axis of the pre-determined reference pose 301. The method further comprises rendering the first reference feature graphic 302 of the first visual state 303 on the first surface 103 coupled to the point of intersection 304 of the reference axis 301 with the first surface 103 and rendering the second reference feature graphic 307 of the first visual state 303 on the second surface 305 coupled to a point of intersection 306 of the reference axis 301 with the second surface 305, wherein the position of the second reference feature graphic 307, the first reference feature graphic 302 and the reference axis 301 is static. The method further comprises rendering the first dynamic feature graphic 310 of a second visual state 311 on the first surface 103 coupled to the point of intersection 308 of the body-fixed axis 300 with the first surface 103 and rendering the second dynamic feature graphic 312 of the second visual state 311 on the second surface 305 coupled to the point of intersection 309 of the body-fixed axis 300 with the second surface 305, wherein the position of the first dynamic feature graphic 310, the second dynamic feature graphic 312 are updated in real time based on the position and the orientation of the physical object 101. The set of feature graphics of the plurality of feature graphics along the reference axis 301 are the first reference feature graphic 302 and the second reference feature graphic 307 and the another set of feature graphics of the plurality of set of feature graphics are the first dynamic feature graphic 310 and the second dynamic feature graphic 312.

The dimension of the first dynamic feature graphic 310 is equal to the dimension of the first reference feature graphic 302 and the dimension of the second dynamic feature graphic 312 is equal to the dimension of the second reference feature graphic 307. Upon intersection of the first reference feature graphic 302 with the first dynamic feature graphic 310 and the second reference feature graphic 307 with the second dynamic feature graphic 312, portions of the intersection 402, 404 are displayed in a third visual state 403 distinct from the first visual state 303 and the second visual state 311. The first visual state 303 is a first colour, the second visual state 311 is a second colour and the third visual state 403 is a third colour. The first visual state of the first reference graphic and the first dynamic feature graphic is the first shape 303, the first visual state of the second reference graphic and the second dynamic feature graphic is the second shape 311 and the modified visual state 403 is the third shape. The position and orientation of the first surface 103 is same as the position and orientation of the real environment object 104. The perspective of the user is tracked and the measurement of the perspective of the user is used for displaying a virtual three-dimensional environment in the same orientation as that of the real environment object 104. The method further comprising updating the orientation of the another set of feature graphics of the plurality of set of feature graphics based on a current position and orientation of the physical object 101.

The method further comprises updating 905 the positions of the another set of feature graphics of the plurality of set of feature graphics based on a current position and orientation of the physical object 101, wherein the another set of feature graphics is dynamic feature graphics that is positionally distributed along the body-fixed axis 300 of the physical object 101 and modifying 906 the visual states of the plurality of set of feature graphics based on the extent of alignment between the body-fixed axis 300 and the reference axis 301. The tracking system 102 provides an input to the three dimensional display device 100 based on the tracking of the position and orientation of the physical object 101 for creating the real-time body-fixed axis 300 and updating the positions of the another set of feature graphics of the plurality of set of feature graphics. The real environment object 104 is spatially tracked and the reference pose is static with respect to the real environment object 104.

The pre-determined reference pose comprises the reference direction non-parallel to the reference axis and/or a reference point on the reference axis. The modification of the visual states of the reference feature graphics and dynamic feature graphics based on the extent of alignment between the body-fixed axis 300 with the reference axis 301, and a body-fixed direction with the reference direction is performed by acquiring the real-time measurement of the predefined body-fixed direction non-parallel to the body-fixed axis 300. The method further comprising acquiring a real-time measurement of a body-fixed point on the body-fixed axis 300, rendering the third reference feature graphic at the reference point along the reference axis 301 comprising an initial visual state, rendering the third dynamic feature graphic coupled to the body-fixed point in an initial visual state, and modifying the visual states of the third reference feature graphic and the third dynamic feature graphic based on the distance between the body-fixed point and the reference point.

The method and the visual guidance system disclosed herein are not limited to a particular computer system platform, processor, operating system, or network. The method and the visual guidance system disclosed herein are not limited to be executable on any particular system or group of systems, and are not limited to any particular distributed architecture, network, or communication protocol.

In an embodiment, the computer programs that implement the methods and algorithms disclosed herein are stored and transmitted using a variety of media, for example, the computer readable media in a number of manners. In an embodiment, hard-wired circuitry or custom hardware is used in place of, or in combination with, software instructions for implementing the processes of various embodiments. Therefore, the embodiments are not limited to any specific combination of hardware and software. The computer program codes comprising computer executable instructions can be implemented in any programming language. Examples of programming languages that can be used comprise C, C++, C#, Java®, JavaScript®, Fortran, Ruby, Perl®, Python®, Visual Basic®, hypertext preprocessor PHP, Microsoft® .NET, Objective-C®, etc. Other object-oriented, functional, scripting, and/or logical programming languages can also be used. In an embodiment, the computer program codes or software programs are stored on or in one or more mediums as object code. In another embodiment, various aspects of the method and the visual guidance system disclosed herein are implemented in a non-programmed environment comprising documents created, for example, in a hypertext markup language HTML, an extensible markup language XML, or other format that render aspects of a graphical user interface GUI or perform other functions, when viewed in a visual area or a window of a browser program. In another embodiment, various aspects of the method and the visual guidance system disclosed herein are implemented as programmed elements, or non-programmed elements, or any suitable combination thereof.

The foregoing examples have been provided merely for the purpose of explanation and are in no way to be construed as limiting of the method and the visual guidance system disclosed herein. While the method and the visual guidance system have been described with reference to various embodiments, it is understood that the words, which have been used herein, are words of description and illustration, rather than words of limitation. Furthermore, although the method and the visual guidance system have been described herein with reference to particular means, materials, and embodiments, the method and the visual guidance system have are not intended to be limited to the particulars disclosed herein; rather, the method and the visual guidance system extend to all functionally equivalent structures, methods and uses, such as are within the scope of the appended claims. While multiple embodiments are disclosed, it will be understood by those skilled in the art, having the benefit of the teachings of this specification, that the method and the visual guidance system disclosed herein are capable of modifications and other embodiments may be effected and changes may be made thereto, without departing from the scope and spirit of the method and the system disclosed herein.

Those skilled in this technology can make various alterations and modifications without departing from the scope and spirit of the invention. Therefore, the scope of the invention shall be defined and protected by the following claims and their equivalents.

FIGS. 1-9 are merely representational and are not drawn to scale. Certain portions thereof may be exaggerated, while others may be minimized. FIGS. 1-9 illustrate various embodiments of the invention that can be understood and appropriately carried out by those of ordinary skill in the art.

In the foregoing detailed description of embodiments of the invention, various features are grouped together in a single embodiment for the purpose of streamlining the disclosure. This method of disclosure is not to be interpreted as reflecting an intention that the claimed embodiments of the invention require more features than are expressly recited in each claim. Rather, as the following claims reflect, inventive subject matter lies in less than all features of a single disclosed embodiment. Thus, the following claims are hereby incorporated into the detailed description of embodiments of the invention, with each claim standing on its own as a separate embodiment.

It is understood that the above description is intended to be illustrative, and not restrictive. It is intended to cover all alternatives, modifications and equivalents as may be included within the spirit and scope of the invention as defined in the appended claims. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein,” respectively.

1. A method for indicating alignment of a body-fixed axis with a reference axis of a pre-determined reference pose, the method comprising:
acquiring a real-time measurement of the body-fixed axis predefined in a coordinate frame of the physical object;
rendering a first surface with an intersection point of the reference axis on the first surface using a three-dimensional display device;
rendering a second surface at an offset from the intersection point of the reference axis present on the first surface;
rendering a plurality of set of feature graphics on the first surface and the second surface in one or more visual states, wherein at least one set of feature graphics of the plurality of set of feature graphics are reference feature graphics that is positionally distributed along the reference axis of the pre-determined reference pose;
updating the positions of another set of feature graphics of the plurality of set of feature graphics based on a current position and orientation of the physical object, wherein the another set of feature graphics is dynamic feature graphics that is positionally distributed along the body-fixed axis of the physical object; and
modifying the visual states of the plurality of set of feature graphics based on the extent of alignment between the body-fixed axis and the reference axis.

2. The method as claimed in claim 1, wherein the step of rendering the plurality of set of feature graphics on the first surface and the second surface comprises:
rendering a first reference feature graphic of a first visual state on the first surface coupled to a point of intersection of the reference axis with the first surface, wherein the position of the first reference feature graphic and the reference axis is static;
rendering a second reference feature graphic of the first visual state on the second surface coupled to a point of intersection of the reference axis with the second surface, wherein the position of the second reference feature graphic is static;
rendering a first dynamic feature graphic of a second visual state on the first surface coupled to a point of intersection of the body-fixed axis with the first surface, wherein the position of the first dynamic feature graphic is updated in real time based on the position and the orientation of the physical object; and
rendering a second dynamic feature graphic of the second visual state on the second surface coupled to a point of intersection of the body-fixed axis with the second surface, wherein the position of the second dynamic feature graphic is updated in real time based on the position and the orientation of the physical object.

3. The method as claimed in claim 2, wherein the set of feature graphics of the plurality of feature graphics along the reference axis are the first reference feature graphic and the second reference feature graphic and the another set of feature graphics of the plurality of set of feature graphics are the first dynamic feature graphic and the second dynamic feature graphic.

4. The method as claimed in claim 1, wherein the step of updating the position and the orientation of the another set of feature graphics of the plurality of set of feature graphics comprises;
providing an input to the three dimensional display device based on the tracking of the position and orientation of the physical object for creating the real-time body-fixed axis and updating the positions of the another set of feature graphics of the plurality of set of feature graphics.

5. The method as claimed in claim 2, wherein the dimension of the first dynamic feature graphic is equal to the dimension of the first reference feature graphic and the dimension of the second dynamic feature graphic is equal to the dimension of the second reference feature graphic.

6. The method as claimed in claim 2, wherein upon intersection of the first reference feature graphic with the first dynamic feature graphic and the second reference feature graphic with the second dynamic feature graphic, portions of the intersection, are displayed in a third visual state distinct from the first visual state and the second visual state.

7. The method as claimed in claim 6, wherein the first visual state is a first colour, the second visual state is a second colour and the third visual state is a third colour.

8. The method as claimed in claim 6, wherein the first visual state is a first shape, the second visual state is a second shape and the third visual state is a third shape.

9. The method as claimed in claim 1, further comprising updating the orientation of the another set of feature graphics of the plurality of set of feature graphics based on a current position and orientation of the physical object.

10. The method as claimed in claim 1, wherein the position and orientation of the first surface is same as the position and orientation of areal environment object.

11. The method as claimed in claim 1, wherein the second surface is transparent.

12. The method as claimed in claim 1, wherein the perspective of the user is tracked and the measurement of the perspective of the user is used for displaying a virtual three-dimensional environment in the same orientation as that of the real environment.

13. The method as claimed in claim 1, wherein the pre-determined reference pose comprises a reference direction non-parallel to the reference axis and/or a reference point on the reference axis.

14. The method as claimed in claim 13, wherein modifying the visual states of the reference feature graphics and dynamic feature graphics based on an extent of alignment between the body-fixed axis with the reference axis, and a body-fixed direction with the reference direction by acquiring a real-time measurement of a predefined body-fixed direction non-parallel to the body-fixed axis.

15. The method as claimed in claim 13, further comprising acquiring a real-time measurement of a body-fixed point on the body-fixed axis;
rendering a third reference feature graphic at the reference point along the reference axis comprising an initial visual state;
rendering a third dynamic feature graphic coupled to the body-fixed point in an initial visual state, and
modifying the visual states of the third reference feature graphic and the third dynamic feature graphic based on the distance between the body-fixed point and the reference point.

16. The method of claim 1, wherein the real environment object is spatially tracked and the reference pose is static with respect to the real environment object.

17. A visual guidance system for indicating alignment of a physical object with a reference axis of a pre-determined reference pose, the visual guidance system comprising one or more processors coupled and configured with components of the visual guidance system for indicating alignment of the physical object with the pre-determined reference pose, the system comprising:
a three-dimensional display device for rendering a first surface with an intersection point of the reference axis on a first surface;
a physical object for performing an action;
a tracking system for tracking the position and orientation of the physical object;
a memory device comprising the reference axis of the pre-determined reference pose;
the three dimensional display device for rendering a body-fixed axis based on the tracked position and orientation of the physical object and a plurality of set of feature graphics on the first surface and the second surface in one or more visual states, wherein at least one set of feature graphics of the plurality of set of feature graphics is positionally distributed along the pre-determined reference pose; and
the three dimensional display device for rendering modified visual states of the plurality of set of feature graphics based on the extent of alignment between the body-fixed axis and the reference axis.

18. The system as claimed in claim 17, wherein the plurality of set of feature graphics on the first surface and the second surface rendered by the three dimensional display device comprises:
a first reference feature graphic of a first visual state on the first surface coupled to the point of intersection of the reference axis with the first surface, wherein the position of the first reference feature graphic and the reference axis is static;
a second reference feature graphic of the first visual state on the second surface coupled to the point of intersection of the reference axis with the second surface, wherein the position of the second reference feature graphic is static;
a first dynamic feature graphic of a second visual state on the first surface coupled to the point of intersection of the body-fixed axis with the first surface, wherein the position of the first dynamic feature graphic is updated in real time based on the position and the orientation of the physical object; and
a second dynamic feature graphic of the second visual state on the second surface coupled to the point of intersection of the body-fixed axis with the second surface, wherein the position of the second dynamic feature graphic is updated in real time based on the position and the orientation of the physical object.

19. The system as claimed in claim 17, wherein the set of feature graphics of the plurality of feature graphics along the reference axis are the first reference feature graphic and the second reference feature graphic and the another set of feature graphics of the plurality of set of feature graphics are the first dynamic feature graphic and the second dynamic feature graphic.

20. The system as claimed in claim 17, wherein the position and orientation of the first surface is same as position and orientation of the real environment object.

21. The system as claimed in claim 17, wherein the second surface rendered by the three dimensional display device is transparent.

22. The system as claimed in claim 17, wherein the tracking system tracks the position and orientation of the three dimensional display device in real time.
